(19) United States
(12) Patent Application Publication
(10) Pub. No.: US 2023/0210655 A1
(43) Pub. Date: Jul. 6, 2023

(54) Accommodating Intraocular Lens with Rigid Tapered Flanges

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 18/007,536

(22) PCT Filed: Jun. 3, 2021
(86) PCT No.: PCT/NL2021/050353

§ 371 Date: Dec. 1, 2022

(30) Foreign Application Priority Data
Jun. 4, 2020 (NL) 2025750

(51) Int. Cl. A61F 2/16

(52) U.S. Cl. CPC A61F 2/1635; A61F 2002/1682

(57) ABSTRACT

Disclosed are accommodating intraocular lenses with a variable power lens and a lens driver coupled to the variable power lens. The driver is arranged to be, at least partially, positioned in an accommodative structure of the eye, for example the sulcus of the eye or the capsular bag of the eye with the driver including a tapered flange which tapers towards its peripheral free end to provide translation of constrictive movement of the accommodative structure in an axial direction into movement onto the variable power lens in a lateral direction.

### CROSS-REFERENCE TO RELATED APPLICATIONS

This application is the United States national phase of International Application No. PCT/NL2021/050353 filed Jun. 3, 2021, and claims priority to The Netherlands Patent Application No. 2025750 filed Jun. 4, 2020, the disclosures of which are hereby incorporated by reference in their entireties.

### BACKGROUND OF THE INVENTION

### Field of the Invention

Accommodating intraocular lenses restore the accommodation of the human eye. Such lenses generally comprise a variable power lens and at least one lens driver.

Description of Related Art Prior art documents include EP1037572, WO0021467, EP1416890 and related documents disclose intraocular lenses with a single optical element which are positioned inside the capsular bag and driven by movement of the capsular bag in a direction perpendicular to the optical axis, in a lateral direction. These lenses differ from the lenses disclosed in the current document which lenses are, in the preferred embodiment, positioned the lens on top of the capsular bag, at the sulcus plane, with the wedge shaped rigid flange moved by constriction of the sulcus in an axial direction with this movement translated into movement of optics into a lateral direction.

EP2547289, and, for example, EP3791827, JP2020124542 and other documents related thereto disclose lenses comprising inflatable optics which can be coupled to inflatable haptics with the haptics partially filled with a fluid communicating with the inflatable optics via a conduit with the ocular structure applying a pumping force on the haptic to cause fluid to flow via said the conduit to the inflatable optics. Such haptics are inflatable, not rigid, and do not move in a lateral direction during the accommodative process while the present invention concerns lenses with rigid, solid, flanges which translate axial movement of an accommodative structure into lateral movement of the haptics during the accommodative process.

US2019269499, US2016030161, EP3791827 and other documents disclosing largely similar designs disclose an accommodative lens coupled to the capsular bag of which the optic is, as excerpted from, in this example US2019269499: ‘deformable such that the inward pressure or outward tension on the optic caused by ciliary muscle contraction or relaxation causes the optic to change shape’ and “in other words, with peripheral force (contraction of the ciliary muscle) and inward movement of the haptics [ . . . ] and with relaxation of the peripheral force (relaxation of the ciliary muscle [ . . . ] and the haptics can be placed outside of the lens capsule and positioned in the ciliary sulcus against the ciliary muscle.” Constriction of the sulcus and translation of axial movement of the ciliary muscle into lateral movement of the haptic plays no role in this lens design in which lateral movement of the ciliary muscle results, directly, in lateral movement of lens components.

WO2009154455, WO2011115860 and numerous documents related thereto disclose accommodating lenses comprising two optical elements positioned at the sulcus plane, on top of the capsular bag which are driven by force by the ciliary mass directed in a lateral direction, forces largely perpendicular to the optical axis. These designs are not suited to and do not mention not illustrate translation of axial movement of the ciliary mass into lateral movement of any lens components.

### SUMMARY OF THE INVENTION

In accommodating lenses disclosed in the present document the lens driver comprises at least one rigid tapered flange, henceforth also: ‘tapered flange’ or ‘flange’ positioned in an accommodative structure of the eye, for example, the sulcus of the eye, the preferred embodiment, or, alternatively, in the rim of the capsular bag. The tapered flange is a wedge shaped tapered flange, which flange tapers towards its peripheral free end. The tapering can have various shapes, for example a linear shape, resulting in a triangular tapering or, alternatively, a curved shape, for example a shape resembling a ‘dolphin's nose’. The preferred embodiment comprising triangular tapering will be used to explain and illustrate the invention.

Such accommodating lens can comprise, for optical functioning, in the preferred embodiment, a radially flexible lens comprising two optical surfaces with the lens providing variable optical power of which the degree of optical power depends on the degree of movement of the mechanical construction in a lateral direction which movement provides a change in the radius of at least one optical surface of the lens. Or, alternatively, the lens can comprise a combination of at least two optical elements with each element comprising at least one largely spherical surface wherein the combination provides variable optical power of which the degree of optical power depends on the degree of movement, in an axial direction, along the optical axis, of the optical elements in opposite directions. Or, alternatively, as disclosed in WO2009154455, WO2011115860 and related documents, incorporated herein by reference, the variable power lens can comprise a combination of at least two optical elements with each element comprising at least one free-form optical surface with the combination of optical surfaces provides variable optical power of which the degree of power depends on the degree of mutual movement in opposite directions of the optical elements in a lateral direction. The free-form optical surfaces can be, for example, smooth cubic optical surfaces.

The variable power lens can also comprise an integrated combination of at least two lenses including at least one, relatively inflexible, fixed power lens and at least one, relatively flexible, variable power lens. All these lenses can be composed of the same lens material, for example, material of which the degree of flexibility depends solely on the degree of cross-linking of the same lens material, by, for example, varying degree of cross-linker material, or, alternatively, applying laser light to selectively alter the degree of cross-linking by intralenticular laser encryption, or, alternatively, the at least two lenses can be manufactured by a layered molding process, or, alternatively, the at least two lenses can be manufactured during a lathing process of a layered lens button. The lens material can be a hydrophilic acrylic lens material, or, alternatively, a hydrophobic acrylic lens material, or, alternatively, the accommodating lens can be composed of a combination of hydrophobic material and hydrophilic material, or, alternatively, any combination of any intraocular materials.

The accommodating lens can be a lens which lens provides both refractive correction of the eye as well as accommodation of the eye. Alternatively, the lens can be an add-on accommodative unit to provide accommodation to an eye of which eye the fixed refraction is corrected by the natural lens, or, alternatively, by any at least one separate fixed optical power artificial intraocular lens which can be positioned in the anterior chamber of the eye, or, alternatively, can be positioned in the posterior chamber of the eye, for example, positioned in the capsular bag of the eye with the add-on accommodative unit in the sulcus.

### DETAILED DESCRIPTION OF THE INVENTION

Note that, firstly, the description of the prior art above is a summary and the explanation of differences between the prior art and the current invention is therefore limited and that, secondly, in the Figures and the description of the Figures, below, positioning of the intraocular lens in the sulcus plane is used to clarify various lens designs and thus this invention not restricted to these particular examples.

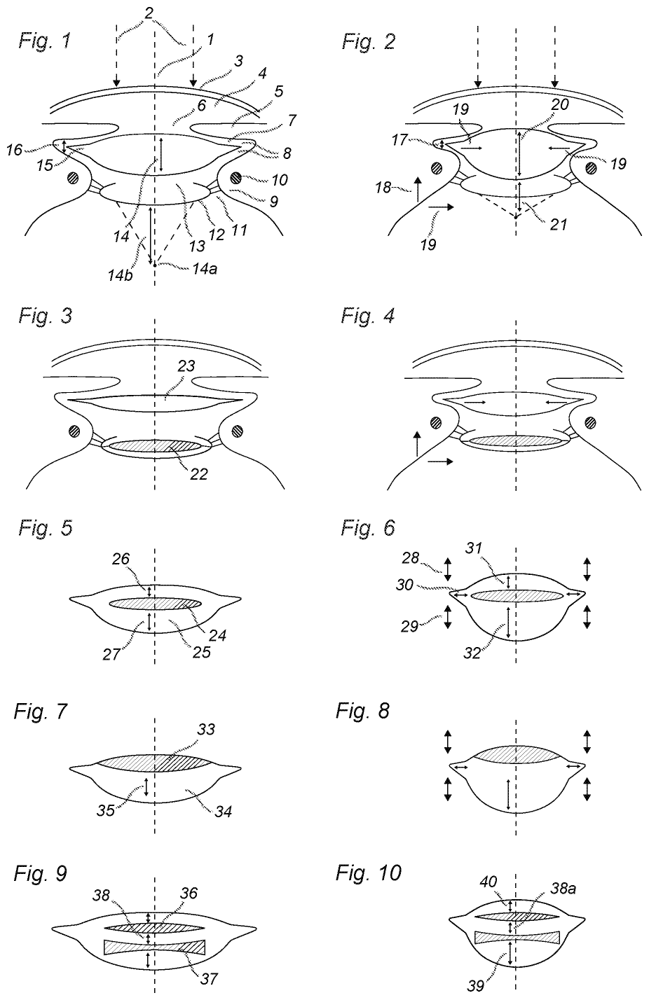

FIGS. 1-2 show, in FIG. 1, a cross section of the eye, with an optical axis, 1, with an, in this example, a parallel incoming light beam, 2, with the eye having a cornea, 3, the anterior chamber of the eye, 4, in front of the iris, 5, the pupil, 6, the posterior chamber of the eye, 7, behind the iris, the sulcus, 8, which sulcus is determined, posteriorly, by the ciliary mass, 9, comprising the ciliary muscle, 10, and determined, anteriorly, by the posterior surface of the iris. The ciliary mass is attached by zonulae, 11, to the capsular bag, 12, of the eye, from which the natural lens is removed by cataract surgery through a capsulorhexis, 13. The accommodating lens comprises a variable power lens, 14, with the optical system of the eye, including the variable power lens, providing a focal spot, 14a, which focal spot moves along the optical axis with the position of the spot depending on the optical power of the variable lens, 14b, which converges the light beam and at least one lens driver, in this example a rigid tapered flange, 15, which flange is positioned, at least partly, in the sulcus. This figure shows a dis-accommodated eye, focusing the eye at far, with a relaxed, opened and backwards retreated ciliary mass and thus with a relatively widened sulcus, 16, with FIG. 2 showing a cross section of the same, now accommodated eye, with a constricted and narrowing, closing, of the sulcus, 17, due to forward movement, an axial movement, 18, of the ciliary mass which closure of the sulcus forces the rigid tapered flange in a lateral direction, 19, in a direction perpendicular to the optical axis, which movement, in turn, provides for an increased thickness, increased radius, of the optical surfaces, 20, of the variable power lens, with the thicker lens providing for an increase in optical power of the variable power lens, 21.

FIGS. 3-4 show, in FIG. 3 (for details refer to FIGS. 1-2), an alternative embodiment of the accommodative lens, in this example with the refractive function, the fixed power optical function, provided by a fixed optical power lens, 22, positioned in the capsular bag and with the accommodative optical power function provided by an add-on accommodative lens, 23. The fixed optical power lens can be an artificial intraocular lens, for example, preferably, a monofocal intraocular lens, or, alternatively, a multifocal intraocular lens, or alternatively, the fixed optical power lens can be a, generally presbyopic, natural lens with FIG. 4 showing the same eye in an accommodated state. So, the lens can be is an add-on accommodative unit to provide accommodation to an eye of which the fixed refraction is corrected by any, at least one, fixed optical power intraocular lens which lens is independent from the add-on accommodative unit. Such fixed power lens can be positioned in the anterior chamber of the eye, or, alternatively, in the posterior chamber of the eye, for example, in the capsular bag of the eye.

FIGS. 5-6 (for details refer to FIGS. 1-2 and FIGS. 3-4) show an alternative embodiment comprising an integrated fixed power section of the lens, 24, comprising, in this example, a section of the same material of which the variable section, 25, with the sections differing, for example, only in the degree of water content providing a relatively inflexible fixed power section providing fixed optical power and a relatively flexible variable section providing variable optical power which section can thickened both anteriorly, 26, and can be thickened posteriorly, 27, with the fixed power section integrated into the variable power lens section with FIG. 6 showing the same lens in an accommodated state with the variable force transferred by the iris, 28, onto the rigid tapered flange and the variable force transferred onto the flange by the ciliary mass, 29 resulting in lateral movement, 30, of the flange, which movement results in increased thickness of the anterior section of the variable power lens section, 31, and in increased thickness of the posterior section of the variable power lens section, 32, which increased thicknesses, in turn, result in variable power lens power of the accommodative lens while the optical power of the fixed power lens remains unaltered. Note that such designs can change the radius of the anterior optical surface, or, the posterior surface, or, both surfaces.

FIGS. 7-8 (for details refer to earlier Figures) show, in FIG. 7, a second alternative embodiment comprising an integrated fixed optical power section of the lens, 33, coupled to the variable section, 34, as in FIGS. 5-6, with, in this embodiment, with the fixed optical power lens section integrated, 35, onto the variable power lens section only at a single optical surface with, in FIG. 8, showing the same lens in accommodated state.

FIGS. 9-10 (for details refer to earlier Figures) show, in FIG. 9, a third alternative embodiment comprising two fixed optical power largely spherical lens sections, in this example a first section providing positive optical power, 36, which section is optically overpowered with regard to the refractive requirements of the eye and a second section providing negative optical power, 37, which section corrects for the overpower of the first section such that the refractive requirements of the eye are met, with the fixed power sections separated by an intra-lenticular space, 38, with, in FIG. 10, the same lens in an accommodated state with variable power provided by thickening of the variable sections, 38a, 39, and by an increase in intra-lenticular space, 40.

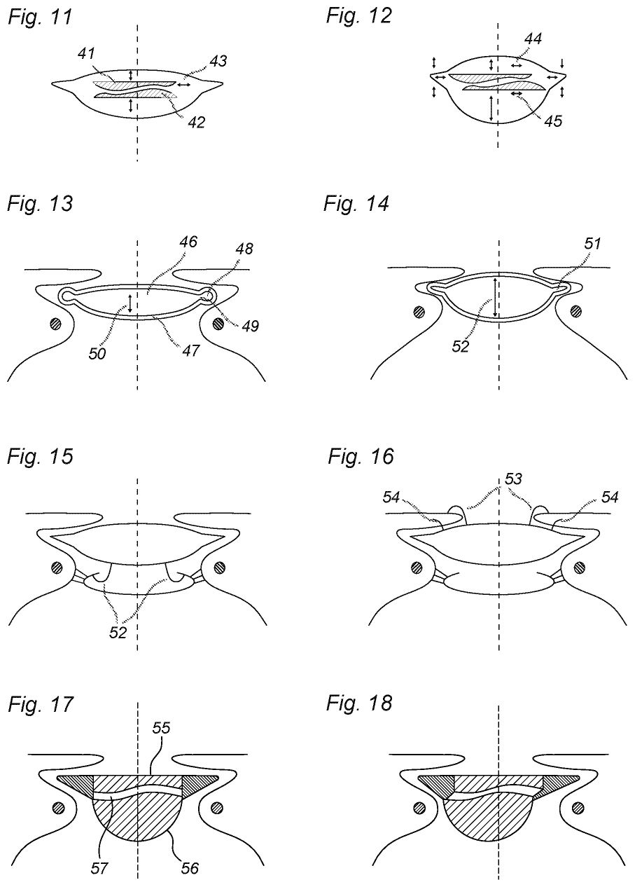

FIGS. 11-12 (for details refer to earlier Figures) show, in FIG. 11, a fourth alternative embodiment comprising two fixed power lenses, 41, 42, with added free-form surfaces which, in combination, provide a variable power lens of which the optical power depends on the degree of mutual shift, 43, of the fixed power lenses, a direction largely perpendicular, laterally, to the optical axis with, in FIG. 12 showing the effect of such said lateral movement, 44, 45, with variable power lens sections functioning as in embodiments described and illustrated in FIGS. 9-12.

FIGS. 13-14 (for details refer to earlier Figures) show, in FIG. 13, an accommodative lens, in a relaxed state, comprising a variable power lens, 46, comprising a fluid filled container, for example, a polymer container with flexible walls, 47, for example a container filled with an oil, with the container coupled to lens drivers, also: bouncing chambers, 48, made of the same material and filled with the same fluid, with the bouncing chambers connected to the variable power lens by fluid channels, 49, with the variable power lens having a thickness, 50, and with, as in FIG. 14, with the variable power lens in an accommodated state, with the flexible bouncing chambers compressed, 51, moving fluids into the variable power lens and thus increasing the thickness, 52, of the variable power lens.

FIGS. 15-16 show additional anchoring of any of the lens embodiments disclosed with in this document with, in FIG. 15, the accommodative lens comprising additional posterior haptics, 52, to anchor the accommodative lens in the capsulorhexis in the capsular bag and, as in FIG. 16, anchoring the accommodative lens by additional anterior haptics, 53, in the pupil or, alternatively, additional haptics, 54, coupling the lens into the iris of the eye. Note that such anchoring in the iris can provide additional accommodative movement of the variable power lens because the iris generally constricts/moves largely in concert with the ciliary mass. The accommodative lens can comprise any combination of said additional haptics. So, the accommodating lens can comprise at least one posterior anchoring component which component provides anchoring of the lens by coupling to the rim of the capsulorhexis in the capsular bag of the eye, or, alternatively, can comprise at least one anterior anchoring component which component provides anchoring of the lens by coupling to the iris of the eye, or, alternatively, can comprise any combination of additional posterior haptics and anterior haptics. FIGS. 17-18 (for details refer to earlier Figures) show, in FIG. 17 an accommodating lens according to, for example, PL1720489, WO2019022608, NL201553, NL2015616 and numerous other disclosures related to such accommodating lenses the accommodating lens in a relaxed state resulting in an emmetrope eye and, in FIG. 18, the same lens in a compressed state resulting in an accommodated eye. The optical elements in both figures comprise, for accommodation, free-form optical surfaces lining the intra-lenticular space, 57. In this example the posterior element, 56, is also fitted with a rotational symmetrical lens to correct for the refraction of the eye. The optical element are shifted in a direction laterally, perpendicular to the optical axis, by movement of the ciliary mass in a direction along the optical axis which movement compresses the sulcus in a direction along the optical axis which compression moves the rigid tapered flange in a direction perpendicular direction to the optical axis, meaning a lateral direction, which movement of the flange, in turn, shifts at least one of the optical elements in a lateral direction.

In summary, this document discloses accommodating lenses for providing accommodation to an eye, with the eye and the lens having the same optical axis, which lenses comprise at least one variable power lens to accommodate the eye, with the variable power lens has a variable optical power, and at least one rigid lens driver coupled to the variable power lens which driver is arranged to be positioned in an accommodative structure of the eye wherein the rigid lens driver comprising at least one tapered flange which tapers towards its peripheral free end to provide translation of constrictive movement of the structure in the eye in an axial direction into movement onto the variable power lens in a lateral direction. The term rigid is used to indicate that the element does not deform upon compression of the accommodative structure of the eye. The compression of the accommodative structure thus may not cause deformation of the rigid component, but causes the rigid component to move or translate, and exert a force onto the lens components.

The accommodative structure in the eye can be the sulcus of the eye, the wedge shaped gap between the anterior surface of the ciliary mass and the posterior surface of the iris, which wedge constricts during the accommodative process and widens during the dis-accommodative process, alternatively, the accommodative structure of the eye can be the ciliary bag of the eye, in eyes from which the natural lens is removed with, in this example, the accommodative structure being the gap between the posterior section of the capsular bag and the remaining rim of the anterior section of the capsular bag post-capsulorhexis, with the posterior section and the remaining rim providing a gap which gap constricts during the accommodative process and widens during the dis-accommodative process. Note that, in all examples provided in this document the intraocular lens regains its original, relaxed, diameter by restoration of the relaxed shape of the intraocular lens by the inherent, outward, elastical properties of the lens driver and/or elastical properties of optical components.

Note that the optical components of the lens can be also implanted at a position in the eye which position differs from the position in which the lens driver is implanted. For example, but not restricted hereto, the optics can be implanted in the capsular bag with the lens driver positioned at the sulcus plane, in front of the capsular bag to allow the variable optics to be driven by the sulcus. Such construction will allow the optics to be driven also when the capsular bag becomes unsuitable for driving a lens because of, for example, hardening or fibrosis or posterior capsule opacification.

The variable power lens can comprise a radially flexible lens comprising two optical surfaces, wherein the degree of optical power of the variable power lens depends on the degree of movement of the lens driver in a lateral direction with the movement providing a change in the radius of at least one optical surface of the lens, or, alternatively, the variable power lens can be a combination of at least two optical elements with each element comprising at least one largely spherical surface, wherein the combination provides variable optical power of which the degree of optical power depends on the degree of movement in an axial direction of the optical elements in opposite directions, or, alternatively, the variable power lens can be a combination of at least two optical elements with each element comprising at least one free-form optical surface, wherein the combination of optical surfaces provides variable optical power of which the degree of power depends on the degree of mutual movement in opposite directions of at least one of the optical elements in a lateral direction.

1. An accommodating intraocular lens, for providing accommodation to an eye, wherein the lens and the eye have the same optical axis, comprising
at least one variable power lens to accommodate the eye and
at least one rigid lens driver coupled to the variable power lens which driver is arranged to be positioned in an accommodative structure of the eye, such as the sulcus;
wherein the rigid lens driver comprises at least one rigid tapered flange which tapers towards its peripheral free end to provide translation of constrictive movement in an axial direction of the accommodative structure in the eye into movement in a lateral direction onto the variable power lens.

2. The lens according to claim 1, wherein the accommodative structure of the eye is the sulcus of the eye, the wedge shaped gap between the anterior surface of the ciliary mass and the posterior surface of the iris, wherein the rigid tapered flange is arranged to be positioned in said accommodative structure at least partially.

3. The lens according to claim 1, wherein the accommodative structure of the eye is the gap between the posterior section of the capsular bag and the remaining rim of the anterior section of the capsular bag.

4. An accommodating lens according to wherein the variable power lens is a combination of at least two optical elements, with each element preferably comprising at least one free-form optical surface, wherein the combination of optical surfaces provides variable optical power of which the degree of power depends on the degree of mutual movement in opposite directions of the optical elements in a lateral direction.

5. The accommodating lens according to claim 1, further comprising at least one posterior anchoring component which component provides anchoring of the lens by coupling to the rim of the capsulorhexis in the capsular bag of the eye.
